(19) United States
(12) Patent Application Publication
(10) Pub. No.: US 2025/0251376 A1
(43) Pub. Date: Aug. 7, 2025

(54) SYSTEMS AND METHODS FOR SEPARATION OF CRISPR/CAS COMPONENTS

(71) Applicant: MRIGlobal, Kansas City, MO (US)

(72) Inventors: Jonathan White, Kansas City, MO (US); Rachel Ginther, Lenexa, KS (US); Kyle Scheel, Kansas City, MO (US)

(21) Appl. No.: 19/037,766

(22) Filed: Jan. 27, 2025

Related U.S. Application Data
(60) Provisional application No. 63/625,663, filed on Jan. 26, 2024.

(51) Int. Cl. G01N 30/96; C12N 9/22; C12N 15/10; G01N 30/74; G01N 30/88

(52) U.S. Cl. CPC G01N 30/96; C12N 9/226; C12N 15/101; G01N 30/74; G01N 2030/8827; G01N 2030/8831

(57) ABSTRACT

Systems, methods, and kits for separating CRISPR/Cas components are provided. The systems, methods, and kits utilize ion exchange chromatography to separate CRISPR/Cas components based on the differing charges of CRISPR-Cas components.

### CROSS-REFERENCE TO RELATED APPLICATIONS

This application claims priority to and the benefit of U.S. Provisional Patent App. Ser. No. 63/625,663 filed on Jan. 26, 2024, entitled “SYSTEMS AND METHODS FOR SEPARATION OF CRISPR/CAS COMPONENTS.” The content of the aforementioned patent application is hereby expressly incorporated by reference in its entirety for all purposes.

### FIELD OF DISCLOSURE

The present disclosure generally relates to systems, methods, and kits for separating CRISPR/Cas components.

### BACKGROUND OF THE INVENTION

The discovery of gene editing and programmable genomic control by clustered regularly interspaced short palindromic repeat (CRISPR) RNAs and their CRISPR-associated (Cas) proteins holds tremendous promise for future therapeutics and curing genetic disease. Recently, the first CRISPR edited cell therapy-based therapeutic was approved for treatment of sickle cell anemia, with other CRISPR-based therapies in the pipeline.

CRISPR-Cas systems are adaptive RNA-guided immune systems present in many bacteria and most archaea. The CRISPR-Cas systems provide bacteria and archaea with a sequence-directed defense against invaders such as viruses and plasmids. A CRISPR locus contains short DNA sequences or fragments, acquired from various invaders. The DNA fragments are transcribed to form CRISPR (cr) RNAs, which guide Cas proteins to cleave complementary invader DNA or RNA in a subsequent infection. For example, in nature, the CRISPR-Cas9 system includes cas9, a RNA-guided DNA endonuclease enzyme, a tracrRNA (trans-activating CRISPR-RNA), and crRNA (CRISPR RNA) guide. In research settings, tracrRNA, and crRNA can be combined into a single guide RNA (sgRNA), where the Cas9 protein forms a ribonucleoprotein (RNP) complex with sgRNA, which introduces double stranded breaks in target DNA.

Despite the growing market for CRISPR-based therapeutics, technologies for comprehensive quality control analysis of CRISPR components and RNP complexes are not well-established. The success of CRISPR gene editing experiments as well as the safety and efficacy of CRISPR drug substances may be increased and improved by thorough quality control assessment of CRISPR reagents and/or by characterization of CRISPR/Cas components. Current systems and methods for characterization of CRISPR/Cas components are limited and do not typically allow for simultaneous characterization of unbound (apo) Cas proteins, unbound CRISPR RNAs (e.g., sgRNA), and/or ribonucleoproteins.

### SUMMARY OF THE INVENTION

A method for separating a sample comprising at least one analyte is provided. The method includes (a) loading a sample comprising at least one analyte onto coupled cation-exchange and anion-exchange columns, where the at least one analyte comprises a CRISPR-Cas9 component selected from the group consisting of a RNA, a protein, a ribonucleoprotein complex, and combinations thereof; and (b) eluting the sample from the coupled cation-exchange and anion-exchange columns using a salt gradient. In further embodiments, the method includes (c) detecting the at least one analyte in the sample. In still further embodiments, the detecting step is performed using a UV detector. In some embodiments, the at least one analyte comprises a CRISPR-Cas9 component selected from the group consisting of Cas9, sgRNA, a ribonucleoprotein complex thereof, and combinations thereof. In certain embodiments, the sample has an ionic strength of at least about 300 mM. In some embodiments, the salt gradient is produced by sodium chloride.

In some embodiments, the sample is eluted with two or more mobile phases and each mobile phase comprises a buffer, a salt, and water. In further embodiments, each of the two or more mobile phases has a pH of about 6 to about 8. In still further embodiments, the two or more mobile phases each have an ionic strength of at least about 300 mM.

In some embodiments, the sample is eluted with a first mobile phase and a second mobile phase. In further embodiments, the first mobile phase has a first concentration of salt and the second mobile phase has a second concentration of salt. In still further embodiments, the first mobile phase and the second mobile phase are in a ratio of about 9:1.

A method for performing ion exchange chromatography on a sample comprising at least one analyte is provided. The method includes (a) contacting a sample with coupled ion exchange columns comprising a cation-exchange column coupled to an anion-exchange column, where the cation-exchange column comprises an immobilized strong cationic stationary phase within an interior of the column and the anion-exchange column comprises an immobilized strong anionic stationary phase within an interior of the column; (b) flowing at least two mobile phases through the immobilized strong cationic stationary phase and the immobilized strong anionic stationary phase, where each of the at least two mobile phases comprises a buffer, a salt, and water; and (c) eluting the at least one analyte from the immobilized strong cationic stationary phase and the immobilized strong anionic stationary phase; where the at least one analyte comprises a CRISPR-Cas9 component selected from the group consisting of a RNA, a protein, a ribonucleoprotein complex, and combinations thereof. In further embodiments, the method includes (d) detecting the at least one analyte in the sample. In still further embodiments, the detecting step is performed using a UV detector. In some embodiments, the at least one analyte comprises a CRISPR-Cas9 component selected from the group consisting of Cas9, sgRNA, a ribonucleoprotein complex thereof, and combinations thereof.

A kit for detecting at least one at least one analyte in a sample is provided. The kit includes (a) an anion-exchange column; (b) a cation-exchange column; (c) a column union to couple the anion-exchange column to the cation-exchange column; (d) a composition containing one or more buffers and a salt; (e) a nuclease-free buffer; and (f) instructions for using the kit to detect the at least one analyte in the sample; where the at least one analyte comprises a CRISPR-Cas9 component selected from the group consisting of a RNA, a protein, a ribonucleoprotein complex, and combinations thereof. In some embodiments, the at least one analyte comprises a CRISPR-Cas9 component selected from the group consisting of Cas9, sgRNA, a ribonucleoprotein complex thereof, and combinations thereof. In certain embodiments, the salt is sodium chloride. In some embodiments, the one or more buffers is selected from the group consisting of Tris (tris(hydroxymethyl)aminomethane), Bis-Tris propane or BTP (1,3-bis(tris(hydroxymethyl)methylamino)propane), and HEPES (4-(2-hydroxyethyl)-1-piperazineethanesulfonic acid), and MES (2-(N-morpholino)ethanesulfonic acid).

### DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein. However, those of skill in the art will understand that the drawings, described below, are for illustrative purposes only. The drawings are not intended to limit the scope of the present teachings in any way.

FIGS. 1A, 1i, and 1C show a schematic representation of (FIG. 1A) positively charged apo-Cas9, (FIG. 1B) negatively charged unbound sgRNA, and (FIG. 1C) an intermediately charged RNP complex.

FIG. 2A shows a chromatogram for apo-Cas9 injected onto coupled strong cation-exchange and strong anion-exchange columns, with a scaled inset showing Cas9 charge variants.

FIG. 2B shows a chromatogram for unbound sgRNA injected onto coupled strong cation-exchange and strong anion-exchange columns, with a scaled inset showing charge heterogeneity of the unbound sgRNA.

FIG. 3 shows a chromatogram for a ribonucleoprotein complex of Cas9 and sgRNA.

FIG. 4A shows a chromatogram produced by using a UV detector having an analytical flow cell to detect components of an eluted sample of humanized mAb on a cation-exchange column.

FIG. 4B shows a chromatogram produced by using a UV detector having an titanium flow cell to detect components of an eluted sample of humanized mAb on a cation-exchange column.

### DETAILED DESCRIPTION

Before any embodiments are described in detail, it is to be understood that the disclosure is not limited in its application to the details of construction and the arrangement of components set forth in the following description or illustrated in the following drawings, which is limited only by the claims that follow the present disclosure. The disclosure is capable of other embodiments, and of being practiced, or of being carried out, in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

As used herein, the term “CRISPR-Cas system” refers to an enzyme system including a guide RNA sequence (e.g., sgRNA, crRNA) that contains a nucleotide sequence complementary or substantially complementary to a region of a target nucleic acid, and a protein with nuclease activity. CRISPR-Cas systems include Type I CRISPR-Cas system, Type II CRISPR-Cas system, Type III CRISPR-Cas system, and derivatives thereof. CRISPR-Cas systems include engineered and/or programmed nuclease systems derived from naturally occurring CRISPR-Cas systems. CRISPR-Cas systems may contain engineered and/or mutated Cas proteins. CRISPR-Cas systems may contain engineered and/or programmed guide RNA. The terms CRISPR-Cas system, CRISPR/Cas system, and CRISPR system are used interchangeably.

The number and of known CRISPR-Cas systems has increased in recent years and the classification of CRISPR-Cas systems has been updated as new systems are discovered. CRISPR/Cas systems can be classified as Types I to VI based on the Cas protein in the system. For example, Cas9 is found in Type II systems, and Cas12 is found in Type V systems. Each Type can be further divided into subtypes. For example, Type II can include subtypes II-A, II-B, and II-C, and Type V can include subtypes V-A and V-B. A recent classification includes 2 classes, 6 types, and 33 subtypes. The CRISPR-Cas systems and Cas nucleases described herein can encompass any Type or variant.

As used herein, the term “sample” refers to a composition potentially containing or suspected of containing at least one analyte or compound of interest, such as a biomolecule, for testing or analysis. For example, the composition may be a fluid composition containing one or more dissolved compounds, such as a mixture of two or more dissolved compounds.

As used herein, “chromatography” refers to a separation method for concentrating or isolating one or more compounds (e.g., biomolecules) found in a sample. Chromatography is a differential migration process. Compounds in a sample traverse a chromatographic column at different rates, leading to their separation. The migration occurs by convection of a fluid phase, referred to as the mobile phase, in relationship to a packed bed of particles or a porous monolith structure, referred to as the stationary phase.

In some types of chromatography, differential migration occurs due to differences in the affinity of analytes with the stationary phase and mobile phase. For example, ion exchange (IEX) chromatography is a type of chromatography in which the analytes in a sample are separated or isolated based on their affinity for a charged stationary phase. Ion exchange chromatography (IEX) separates analytes with differences in surface charge, where the separation is based on the reversible interaction between a charged analyte and an oppositely charged stationary phase, e.g., a resin. The net surface charge of some analytes, such as proteins, varies according to the surrounding pH. The pH at which a protein has no net charge is called isoelectric point (pI). Above its isoelectric point (pI), a protein will bind to a positively charged anion exchanger. Below its pI, a protein will bind to a negatively charged cation exchanger. Analytes bind as they are loaded onto a column. The conditions are then altered so that bound substances are desorbed differentially. Elution is usually performed by increasing salt concentration or changing pH in a gradient.

Additionally, while the following discussion may describe features associated with specific devices or embodiments, it is understood that additional devices and/or features can be used with the described systems and methods, and that the discussed devices and features are used to provide examples of possible embodiments, without being limited.

System and Method for Separating apo-Cas, unbound sgRNA, and RNP

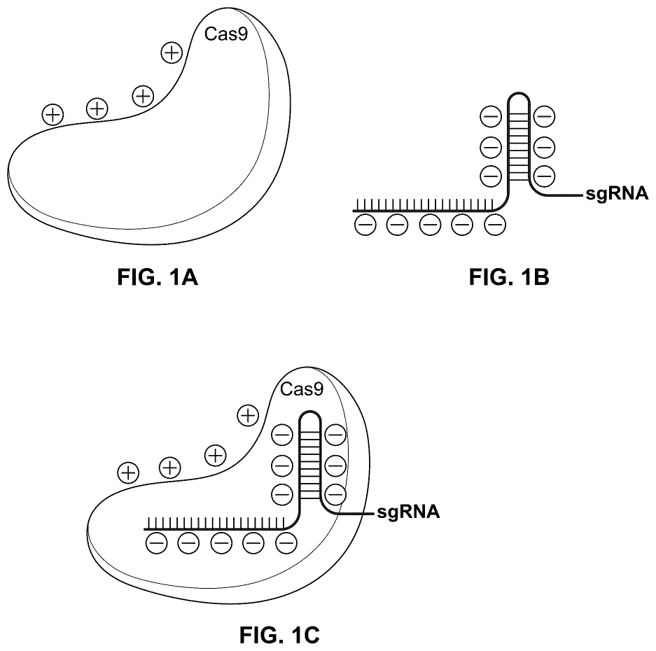

Recently, the first CRISPR edited cell therapy-based therapeutic was approved for treatment of sickle cell anemia, with other CRISPR-based therapies in the pipeline. However, despite the growing market for CRISPR-based therapeutics, technologies for comprehensive quality control analysis of CRISPR components, including ribonucleoprotein (RNP) complexes, are not well-established. To ensure safe and effective CRISPR drug substances, thorough quality control assessment of CRISPR components is needed. Current methods for characterization of CRISPR-Cas components are limited. For example, current methods do not typically allow for simultaneous characterization of multiple CRISPR-Cas components (e.g., apo-Cas, unbound sgRNA, and RNP species). By leveraging the differing charges of CRISPR-Cas components, such as apo-Cas, unbound sgRNA, and RNP, separation of these components may be achieved using ion exchange chromatography. For example, under native conditions, Cas9 carries a positive charge, sgRNA is highly negatively charged, and, consequently, the RNP complex exhibits an intermediate charge state, as shown in FIG. 1.

Ion exchange chromatography is known and has been used to separate various analytes, including biomolecules, such as proteins and nucleic acids, based on their ionic interactions with oppositely charged moieties present on a stationary phase. In cation-exchange chromatography, positively charged proteins adsorb to a negatively charged stationary phase, while in anion-exchange chromatography negatively charged proteins adsorb to a positively charged stationary phase. The adsorbed proteins can be made to elute via a salt gradient in a mobile phase. It has been surprisingly discovered that apo-Cas9, unbound sgRNA, and a RNP complex containing Cas9 and sgRNA can be separated in a single experimental run using coupled ion exchange chromatography under selected conditions, such as the ionic strength of the mobile phase(s). Thus, an all-in-one method for the separation and/or analysis of CRISPR components and the characterization of ribonucleoprotein complex formation is provided. This improved method and system for separating apo-Cas9, unbound sgRNA, and RNP complexes thereof provides a number of benefits, such as allowing for the characterization of RNP formation, allowing for the stoichiometry of the overall mixture to be monitored, allowing for charge variants of apo-Cas9 and unbound sgRNA to be characterized.

A system and method for separating one or more analytes comprising CRISPR-Cas components, including but not limited to Cas proteins, CRISPR RNA, and ribonucleoproteins (RNPs), are thus provided. As used herein, “components” of a CRISPR-Cas system include naturally occurring, engineered, and/or mutated Cas proteins, CRISPR RNA, such as tracrRNA, crRNA, and/or sgRNA, as well as any molecule that may be employed in gene editing using a CRISPR-Cas system. Such molecules may include therapeutic agents, intermediates, reagents, and any other component used in CRISPR gene editing, including various proteins, ribonucleotides, fragments thereof, constructs thereof, and complexes thereof, such as RNP complexes.

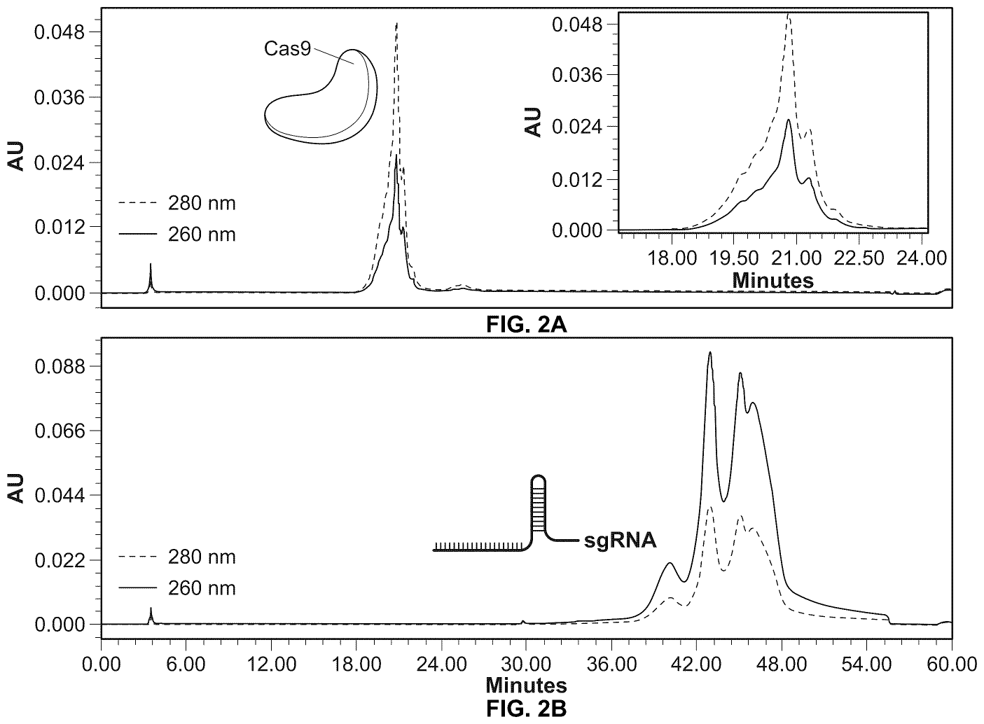

In some aspects, the analyte comprises a Cas protein, a CRISPR RNA, a target nucleic acid, or a RNP complex thereof. The Cas protein may be selected from the group consisting of cas9, cas12 (e.g., cas12a), cas13 (e.g., cas13a), and cas14. The CRISPR RNA may be selected from the group consisting of tracrRNA, crRNA, and sgRNA. Some CRISPR RNA analytes, such as sgRNA, may exhibit surface charge heterogeneity. Advantageously, the system and/or method disclosed herein may show charge heterogeneity that is unseen by other systems and/or methods. In some aspects, the system or method disclosed herein may be used to show charge heterogeneity in CRISPR components, such as sgRNA. For example, in FIG. 2, charge variants are resolved on apo-Cas9, and significant charge heterogeneity is revealed for unbound sgRNA.

In certain aspects, the analyte comprises cas9 protein, such as apo-Cas9, sgRNA, such as unbound sgRNA, or a RNP complex thereof. The cas9 may be based on Streptococcus pyogenes cas9 or Staphylococcus aureus cas9. The one or more analytes may be present in a sample that includes unwanted components and the sample may thus be filtered, concentrated, enriched, or otherwise processed prior to separation.

The system and method may comprise coupled ion exchange columns. The ion exchange columns may be coupled via a column union. Ion exchange columns include cation-exchange columns, such as strong cation-exchange (SCX) and weak cation-exchange (WCX), and anion-exchange columns, such as strong anion-exchange (SAX), and weak anion-exchange (WAX) columns. The coupled cation- and anion-exchange columns may allow for the simultaneous retention of a Cas protein, such as Cas9, on the cation-exchange column and CRISPR RNA, such as sgRNA, on the anion-exchange column. In some aspects, various chromatographic parameters may be optimized to achieve separation between different CRISPR components (optionally, this may occur after column coupling).

In certain aspects, the system and method comprise a weak anion-exchange column coupled to a strong cation-exchange column. For example, the system or method may include a strong cation exchange column (e.g., Waters BioResolve SCX, 3 μm, 4.6×50 mm, P/N 186009058) coupled with a weak anion exchange column (e.g., Waters GenPak FAX, 2.5 μm, 4.6×100 mm, P/N WAT015490). In some aspects, the system and method comprise a strong anion-exchange column coupled to a strong cation-exchange column. Without being bound by theory, it is believed that a strong anion exchange column may provide sharper sgRNA peaks and better resolution from certain elution windows, such as the Cas9 elution window. Suitable commercially available strong anion-exchange columns include the Protein-Pak Hi-Res Q, 100×4.6 mm, 5 μm column (Waters Corporation, Milford, MA, USA), Agilent Bio SAX, NP5, 50×4.6 mm, 5 μm column (Agilent, Santa Clara, CA, USA), Agilent PL-SAX 1000 Å, 4.6×50 mm, 5 μm (Agilent, Santa Clara, CA, USA), Hypersil GOLD™ SAX, 100×4.6 mm, 5 μm column (Thermo Fisher Scientific, USA). Other known strong anion-exchange columns are also suitable for performing separations according to the disclosure. Suitable commercially available strong anion-exchange columns include the BioResolve SCX mAb, 50×4.6 mm, 3 μm column, (Waters Corporation, Milford, MA, USA). Other known cation-exchange columns, including Thermo Scientific MAb Pac SCX, Thermo Scientific Pro Pac SCX, Thermo Scientific Pro Pac WCX, Thermo Scientific Pro Pac Elite WCX, Phenomenex BioZen WCX, Agilent Bio SCX, Agilent Bio WCX, Sepax Proteomix SCX, Sepax Proteomix WCX, Sepax Antibodix WCX, Tosoh TSKgel SP-STAT, Tosoh TSKgel SP-NPR, and YMC BioPro SP-F, are also suitable for performing separations according to the disclosure.

The size of the columns can be selected based on factors such as the amount of sample to be analyzed. For example, for analyzing small amounts of sample, a microbore column, a capillary column, or a nanocolumn may be used. In some instances, the column may be porous. The columns may be equipped with high performance surfaces, such as MaxPeak™ High Performance Surfaces (Waters Corporation, Milford, MA, USA), to mitigate the loss of metal-sensitive analytes. High performance surfaces, in contrast to traditional stainless-steel column hardware, may provide an organic/inorganic barrier between the analyte and metal surface that functions to mitigate nonspecific adsorption.

The system and method may comprise a high-performance liquid chromatography (HPLC) system or ultra-performance liquid chromatography (UPLC) system. Generally, UPLC systems operate at higher pressures (up to 15,000 psi) than HPLC systems (up to 6,000 psi) and use smaller particles in their columns, which improves resolution and sensitivity. UPLC is particularly suitable for more complex samples, like biological samples. Suitable UPLC systems include the commercially available ACQUITY™ UPLC™ H-Class Bio System (Waters Corporation, Milford, MA, USA) and the ACQUITY™ Premier System with a Binary Pump and FTN-SM.

The system or method of the disclosure may comprise a detector for detecting one or more analytes in a sample (e.g., eluted sample). As used herein, “detecting” includes qualitative detection (e.g., presence or absence of an analyte without quantification), semi-quantitative detection (e.g., presence or absence of a specified amount of analyte), quantitative detection (e.g., quantifying the amount of analyte), and/or differentiation of multiple analytes. The detector may comprise a flow cell. Suitable detectors include a refractive index detector, a UV/Vis (ultraviolet/visible) wavelength light detector, a diode array detector, a light-scattering detector, and combinations thereof. In some aspects, the system or method comprises a non-destructive detector, such as a UV/Vis (ultraviolet/visible) wavelength light detector, which may be used to detect components of the eluted sample and produce a chromatogram. In certain aspects, the system or method comprises a UV detector. In certain aspects, the system or method comprises a corrosion resistant instrument equipped with UV detection. In some aspects, the system or method comprises low adsorption flow path components. In certain aspects, the system or method comprises a corrosion resistant instrument equipped with UV detection and low adsorption flow path components.

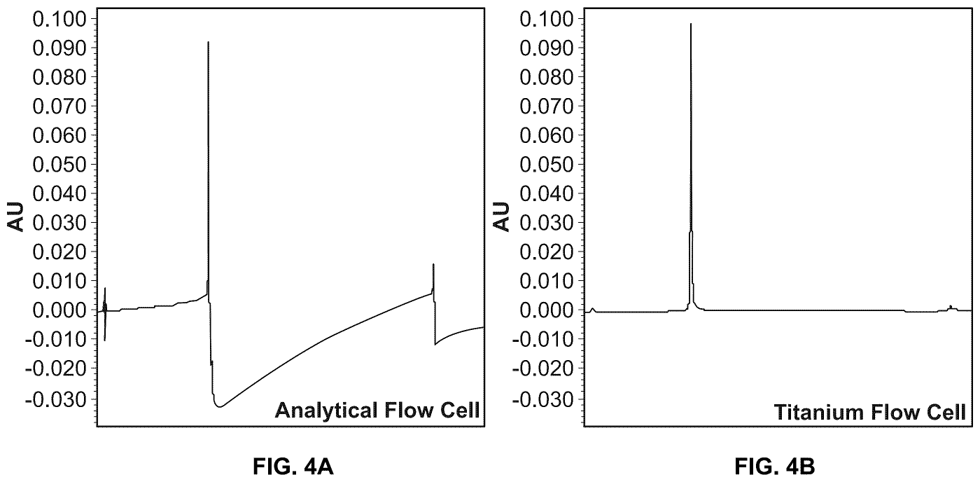

In some aspects, the system or method comprises a UV detector with a titanium flow cell. The typical analytical flow cells in a UPLC-UV detector may be incompatible with proteins, such as the Cas protein, under ion exchange conditions. In particular, the combination of a high ionic strength mobile phase and a traditional flow cell coating may cause protein-flow cell interactions. Without being bound by theory, when an analyte comprises a protein, such as a Cas protein, it is believed that a titanium (or other inert metal) flow cell may reduce or prevent protein-flow cell interaction, as shown in FIG. 4.

Suitable UV detectors include the commercially available ACQUITY™ Premier TUV Detector with titanium flow cell (5 mm) (Waters Corporation, Milford, MA, USA).

The coupled ion exchange columns may be placed in fluid communication with the detector, which can detect the change in the nature of the mobile phase as the mobile phase exits the column. The detector may register and record these changes as a plot, referred to as a chromatogram, which may be used to determine the presence or absence of the analyte and/or the concentration of the analyte. The time at which an analyte leaves an ion exchange column, referred to as the retention time, is an indication of the charge of the analyte and its affinity to the stationary phase of the column.

Thus, in some aspects, a system or method for separating CRISPR/Cas components is provided, where the system or method includes the use of ion exchange (IEX) high-performance liquid chromatography (HPLC) with ultraviolet (UV) detection. The method may include coupling of an anion exchange column and a cation exchange column, allowing for simultaneous retention of Cas protein, e.g., Cas9, on the cation exchange column and sgRNA on the anion exchange column. The system or method may include the application of a corrosion resistant instrument equipped with UV detection, low adsorption flow path components, a titanium flow cell, and/or columns equipped with high performance surfaces. The system and/or method described herein may also be combined with other known analytical method(s) to provide additional information about an analyte.

The system or method may include the use of at least two mobile phases or eluents, and optionally three or more mobile phases or eluents. As discussed above, the system or method may involve the use of coupled ion exchange chromatography under selected and/or optimized conditions, such as the pH and the ionic strength of the mobile phase(s). The pH and ionic strength of each mobile phase may be selected and optimized to improve analyte solubility while allowing for sufficient retention of an analyte on a column and resolution of analyte charge heterogeneity. In particular, the pH and ionic strength of each mobile phase may be optimized to provide sufficient retention of Cas protein, e.g., Cas9, on the cation-exchange column while also ensuring solubility of the Cas protein. In some aspects, each of the mobile phases has a pH ranging from about 6 to about 8, or about 6.5 to about 7.5, or about 6.5.

Each of the mobile phases may comprise a buffering agent, a salt, an optional organic solvent, and water. In some aspects, each of the mobile phases comprises a buffer or buffering agent selected from the group consisting of Tris (tris(hydroxymethyl)aminomethane), Bis-Tris propane or BTP (1,3-bis(tris(hydroxymethyl)methylamino)propane), and HEPES (4-(2-hydroxyethyl)-1-piperazineethanesulfonic acid), and MES (2-(N-morpholino)ethanesulfonic acid). Each of the mobile phases may comprise an optional organic solvent, such as methanol, acetonitrile, and ethanol. In certain aspects, each of the mobile phases comprises a buffering agent selected from the group consisting of Tris and Bis-Tris propane. Tris and Bis-Tris propane allow for the pH and ionic strength of the mobile phase to be readily optimized for the separation of Cas proteins, such as Cas9. In some aspects, each of the mobile phases comprises Bis-Tris propane.

As indicated above, some CRISPR-Cas components, such as Cas protein (e.g., Cas9) and CRISPR-Cas RNP (e.g., CRISPR-Cas9 RNP), are less soluble, or even insoluble, in water and low ionic strength solutions. For example, an ionic strength of at least about 300 mM is believed to be optimal for dissolving Cas9. Thus, the concentration of salt in each of the mobile phases may be selected such that the ionic strength of the mobile phase is high enough to solubilize a Cas protein. The starting ionic strength of the ion exchange gradients may also be adjusted to be sufficiently high to ensure retention of Cas protein. Generally, the starting ionic strength may be optimized depending on the sample type and column type used. Additionally, an analyte may be diluted prior to injection of the analyte onto an ion exchange column and, in the case of a less soluble or insoluble analyte, such as Cas protein (e.g., Cas9), the diluent may be selected to ensure that the analyte is solubilized or resolubilized. For example, Cas9 may be diluted in or combined with a diluent having a salt concentration that is sufficient to achieve a selected ionic strength and solubilize the analyte. In some aspects, a Cas9 protein may be diluted in a mobile phase comprising a salt at a concentration that is selected such that the ionic strength of the mobile phase is high enough to solubilize a Cas protein. In certain aspects, the ionic strength of the mobile phase is at least 300 mM or from about 300 mM to about 500 mM.

The salt and/or the concentration of the salt may be selected to create an optimal salt gradient and/or to improve the separation of the CRISPR components. In some aspects, the salt is selected from the group consisting of chloride-containing salts and bromide-containing salts. In certain aspects, the salt is a chloride-containing salt, such as sodium chloride. The salt gradient used to elute the sample need not be linear. For example, nonlinear, multi-linear, and multi-isocratic gradients may also be used. The gradient selected for the elution may be optimized to achieve the highest resolution for a particular sample. Each mobile phase may comprise a different concentration of salt or one mobile phase may be free of salt. The salt gradient may be formed by varying the ratio of one mobile phase to another mobile phase, for example varying the ratio of a first mobile phase to a second mobile phase. In some aspects, two mobile phases are used, where a first mobile phase comprises a first concentration of salt(s) and a second mobile phase comprises a second concentration of salt(s). To create the salt gradient, the ratio of first mobile phase to second mobile phase run through the coupled ion exchange columns may be varied during the elution step.

In some instances, the system and/or method may include a first mobile phase solution comprising 10 mM Tris in water with a pH of about 7.5; 10 mM Tris, 150 mM NaCl in water with a pH of about 7.5; 10 mM Bis-Tris propane (BTP), 20 mM NaCl, 5% acetonitrile in water with a pH of about 6.5; 10 mM HEPES, 20 mM NaCl in water with a pH of about 7.5; 10 mM BTP, 20 mM NaCl in water with a pH of about 6.5; 10 mM BTP, 150 mM NaCl in water with a pH of about 6.5; or 10 mM BTP, 150 mM NaCl in water with a pH of about 7.5.

In some instances, the system and/or method may include a second mobile phase solution comprising: 10 mM Tris, 1 M NaCl in water with a pH of about 7.5; 10 mM Tris, 1 M NaCl in water with a pH of about 7.5; 10 mM BTP, 1 mM NaCl, 5% acetonitrile in water with a pH of about 6.5; 10 mM HEPES, 1 M NaCl in water with a pH of about 7.5; 10 mM BTP, 1 M NaCl in water with a pH of about 6.5; 10 mM BTP, 1M NaCl in water with a pH of about 6.5; or 10 mM BTP, 1M NaCl in water with a pH of about 7.5.

In some instances, the system and/or method may include a first mobile phase solution comprising 10 mM BTP, 150 mM NaCl in water with a pH of about 6.5 and a second mobile phase solution comprising 10 mM BTP, 1 M NaCl in water with a pH of about 6.5. In certain instances, the first mobile phase solution is mixed with the second mobile phase solution at a ratio of about 9:1.

The reagents and materials used in the system and/or method disclosed herein may be compiled into a kit for ease of use. Thus, a kit for detecting a CRISPR-Cas component in a test sample is also provided. In various aspects, the kit may include an anion-exchange column, a cation-exchange column, a column union to couple the anion-exchange column to the cation-exchange column, a composition containing one or more buffers and, optionally, a salt, (e.g., a concentrated composition that can be readily diluted to prepare one or more mobile phases), a nuclease-free buffer (for diluting or resuspending a CRISPR-Cas component), or mixtures thereof. The buffers, reagents, and/or materials included in a kit may be optimized as described herein. The kit may also include protocols or instructions for using the buffers, reagents, and/or materials in the kit for performing the test method. The protocols or instructions may reflect the optimized conditions and materials described herein.

It will be appreciated by those skilled in the art that while the above disclosure has been described above in connection with particular embodiments and examples, the above disclosure is not necessarily so limited, and that numerous other embodiments, examples, uses, modifications and departures from the embodiments, examples and uses are intended to be encompassed by the claims attached hereto.

### EXAMPLES

### Sample Preparation

### Example 1—Coupled Ion Exchange Chromatography for Separating apo-Cas9, unbound sgRNA, and a RNP Complex Including Cas9 and sgRNA

A sample containing apo-Cas9, a sample containing unbound sgRNA, and a sample containing a RNP complex including Cas9 and sgRNA are each separated using coupled ion exchange columns and a sodium chloride gradient at a column temperature of about 20° C. to about 25° C. (ambient temperature). The temperature of each sample is 8° C.

The sample containing apo-Cas9 is prepared by mixing a Cas9 nuclease protein buffered in glycerol at a concentration of 61.8 μM (Horizon Discovery, Cambridge, UK) in a 1:1 (v:v) ratio with mobile phase B, which is described below, to form a solution, which is then diluted using nuclease-free Tris EDTA buffer (Integrated DNA Technologies, Coralville, Iowa) to prepare a ˜3.7 μg/μL (22.9 μM) solution of Cas9.

The sample containing sgRNA is made by resuspending Human HRPT1 sgRNA (GenScript, Piscataway, NJ) in nuclease-free Tris EDTA buffer (Integrated DNA Technologies, Coralville, Iowa) to prepare a ˜1 μg/μL (24.1 μM) solution of sgRNA.

The sample containing a RNP complex including Cas9 and sgRNA is prepared by mixing a solution of sgRNA, which is prepared by resuspending Human HRPT1 sgRNA (GenScript, Piscataway, NJ) in nuclease-free Tris EDTA buffer (Integrated DNA Technologies, Coralville, Iowa) to form a 93 μM solution, with a solution of Cas9, which is prepared by mixing a Cas9 nuclease protein buffered in glycerol at a concentration of 61.8 μM (Horizon Discovery, Cambridge, UK) in a 1:1 (v:v) ratio with mobile phase B, which is described below, to form a 30.9 μM solution. The solution of sgRNA is added to the solution of Cas9 so as to achieve a slight stoichiometric excess of sgRNA (final concentrations of 24.1 μM sgRNA and 22.9 μM Cas9). The solution of sgRNA is complexed with the solution of Cas9 at room temperature for 30 minutes prior to analysis.

The cation-exchange column is a BioResolve SCX mAb, 50×4.6 mm, 3 μm column (Waters Corporation, Milford, MA, USA) and the anion-exchange column is a Protein-Pak Hi-Res Q, 100×4.6 mmm, 5 μm column (Waters Corporation, Milford, MA, USA). The cation-exchange column is coupled to the anion exchange column via a column union (available as Part No. 700009524 from Waters Corporation, Milford, MA, USA). The coupled columns are used with a flow rate of 0.24 mL/min. Two mobile phases, mobile phase A and mobile phase B, are used in the separation. Mobile phase A contains 10 mM Bis-Tris propane and 150 mM NaCl, is filtered through a 0.2 μm nylon membrane filter, and has a pH of 6.5 in water. Mobile phase B contains 10 mM Bis-Tris propane and 1 M NaCl, is filtered through a 0.2 μm nylon membrane filter, and has a pH of 6.5 in water. 10 μl of a sample comprising 229 pmol apo-Cas9, 241 pmol unbound sgRNA, or a combination of 12 pmol unbound sgRNA and 229 pmol RNP is injected onto the coupled ion exchange columns. Each sample is eluted from the coupled ion exchange columns by varying the percentage of mobile phase A and mobile phase B over the course of 60 minutes as shown in the gradient table (Table 2) below.

| Time (min) | % Mobile Phase A | % Mobile Phase B |
|---|---|---|
| Initial | 90 | 10 |
| 52.0 | 0 | 100 |
| 52.1 | 90 | 10 |
| 60.0 | 90 | 10 |

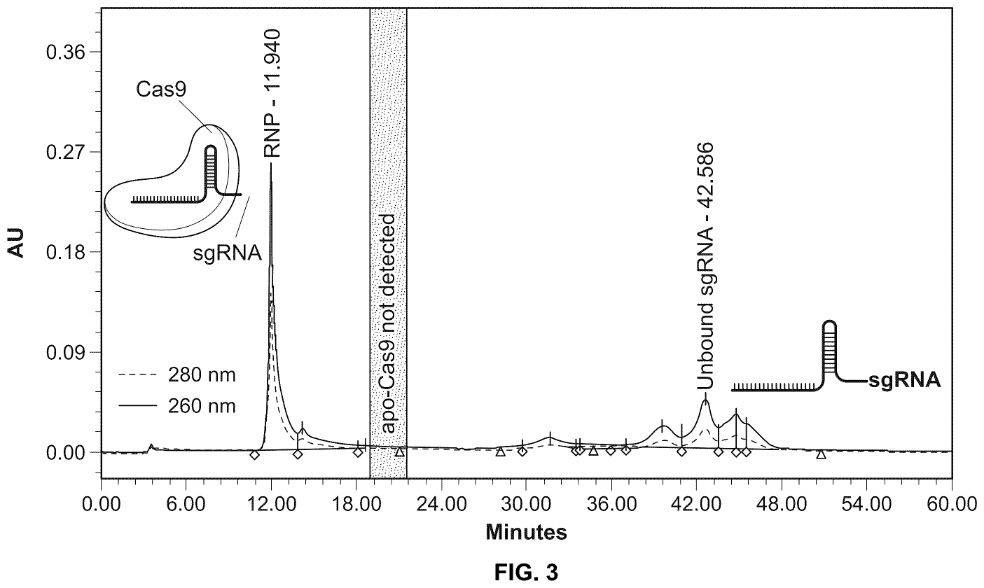

The eluted sample is detected by an ACQUITY™ Premier TUV Detector with a titanium flow cell at 260 nm and 280 nm. The chromatograms obtained from the separations are shown in FIGS. 2 and 3. As shown in the FIG. 2, apo-Cas9 protein is retained on the cation-exchange column and unbound sgRNA is retained on the anion-exchange column. Some charge variants are observed in the apo-Cas9 sample, while significant charge heterogeneity is revealed in the unbound sgRNA sample. As shown in FIG. 3, a sharp, homogenous peak is present at approximately 12 minutes, indicating RNP complexation. The peak corresponding to apo-Cas9 at approximately 20 minutes is not detected, indicating complete complexation of Cas9. The heterogeneous unbound sgRNA eluting from approximately 40 to 48 minutes is not completely consumed, as is expected given the slight excess of sgRNA in solution.

1. A method for separating a sample comprising at least one analyte, the method comprising:
(a) loading the sample comprising at least one analyte onto coupled cation-exchange and anion-exchange columns, wherein the at least one analyte comprises a CRISPR-Cas9 component selected from the group consisting of a RNA, a protein, a ribonucleoprotein complex, and combinations thereof; and (b) eluting the sample from the coupled cation-exchange and anion-exchange columns using a salt gradient.

2. The method of claim 1, wherein the at least one analyte comprises a CRISPR-Cas9 component selected from the group consisting of Cas9, sgRNA, a ribonucleoprotein complex thereof, and combinations thereof.

3. The method of claim 1, wherein the salt gradient is produced by sodium chloride.

4. The method of claim 1, wherein the sample is eluted with two or more mobile phases and each mobile phase comprises a buffer, a salt, and water.

5. The method of claim 4, wherein the sample is eluted with a first mobile phase and a second mobile phase.

6. The method of claim 5, wherein the first mobile phase has a first concentration of salt and the second mobile phase has a second concentration of salt.

7. The method of claim 4, wherein the two or more mobile phases each have a pH of about 6 to about 8.

8. The method of claim 4, wherein the two or more mobile phases each have an ionic strength of at least about 300 mM.

9. The method of claim 1, wherein the sample has an ionic strength of at least about 300 mM.

10. The method of claim 1, wherein the method further comprises detecting the at least one analyte in the sample.

11. The method of claim 10, wherein the detecting is performed using a UV detector.

12. A method for performing ion exchange chromatography on a sample comprising at least one analyte, the method comprising:
a. contacting said sample with coupled ion exchange columns comprising a cation-exchange column coupled to an anion-exchange column, wherein the cation-exchange column comprises an immobilized strong cationic stationary phase within an interior of the column and the anion-exchange column comprises an immobilized strong anionic stationary phase within an interior of the column;
b. flowing at least two mobile phases through the immobilized strong cationic stationary phase and the immobilized strong anionic stationary phase, wherein each of the at least two mobile phases comprises a buffer, a salt, and water; and
c. eluting the at least one analyte from the immobilized strong cationic stationary phase and the immobilized strong anionic stationary phase;
wherein the at least one analyte comprises a CRISPR-Cas9 component selected from the group consisting of a RNA, a protein, a ribonucleoprotein complex, and combinations thereof.

13. The method of claim 12, wherein the method further comprises detecting the at least one analyte in the sample.

14. The method of claim 13, wherein the detecting is performed using a UV detector.

15. A kit for detecting at least one at least one analyte in a sample, the kit comprising:
a. an anion-exchange column;
b. a cation-exchange column;
c. a column union to couple the anion-exchange column to the cation-exchange column;
d. a composition containing one or more buffers and a salt;
e. a nuclease-free buffer; and
f. instructions for using the kit to detect the at least one analyte in the sample;
wherein the at least one analyte comprises a CRISPR-Cas9 component selected from the group consisting of a RNA, a protein, a ribonucleoprotein complex, and combinations thereof.

16. The kit of claim 15, wherein the at least one analyte comprises a CRISPR-Cas9 component selected from the group consisting of Cas9, sgRNA, a ribonucleoprotein complex thereof, and combinations thereof.

17. The kit of claim 15, wherein the salt is sodium chloride.

18. The kit of claim 15, wherein the one or more buffers is selected from the group consisting of Tris (tris(hydroxymethyl)aminomethane), Bis-Tris propane or BTP (1,3-bis(tris(hydroxymethyl)methylamino)propane), and HEPES (4-(2-hydroxyethyl)-1-piperazineethanesulfonic acid), and MES (2-(N-morpholino)ethanesulfonic acid).

19. The kit of claim 15, wherein the anion-exchange column is a strong anion-exchange column.

20. The kit of claim 15, wherein the cation-exchange column is a strong cation-exchange column.
